(19) United States
(12) Patent Application Publication
(10) Pub. No.: US 2009/0035364 A1
(43) Pub. Date: Feb. 5, 2009

(54) PARA-SUBSTITUTED 2-ALKOXYPHENOL COMPOUNDS

(76) Inventors: Christophe C. Galopin, Chesterfield, VA (US); Stefan Michael Furrer, Cincinnati, OH (US); Jay Patrick Slack, Loveland, OH (US); Pablo Victor Krawec, Cincinnati, OH (US); Lucienne Cole, Cincinnati, OH (US)

Correspondence Address: CURATOLO SIDOTI CO., LPA, 24500 CENTER RIDGE ROAD, SUITE 280, CLEVELAND, OH, 44145, US

(21) Appl. No.: 12/282,349

(22) PCT Filed: Mar. 13, 2007
(86) PCT No.: PCT/CH07/00135

§ 371 Date: Sep. 24, 2008

Related U.S. Application Data
(60) Provisional application No. 60/782,466, filed on Mar. 15, 2006.

(51) Int. Cl. A61K 9/127; C07C 211/45; A61K 31/136; A61K 36/534; A61K 36/61; A61K 9/68

(52) U.S. Cl. 424/450; 564/426; 514/656; 424/747; 424/742; 424/48

(57) ABSTRACT

Para-substituted 2-alkoxyphenols having cooling properties of formula (I) wherein R¹ is methyl or ethyl; Y is, NH, O or S; R² and R³ are independently of each other hydrogen or C₁₋₃ alkoxy; n is 0 or 1; and the dotted line between C-2 and C-2′ represents at least one of no bond, a single bond, or a (CH₂)ₘ— group, wherein m is 1 or 2. A process for their production and consumer products that have or use them are also disclosed.

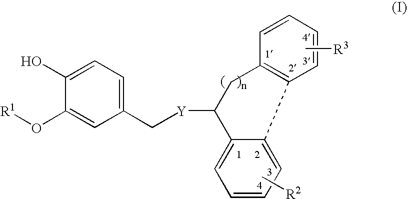

The present invention relates to para-substituted 2-alkoxyphenols having cooling properties. The present invention refers furthermore to a process for their production and to consumer products comprising them.

In the flavor and fragrance industry there is an ongoing demand for compounds having unique cooling properties that provide the user with a pleasing cooling effect and which are suitable for use in a variety of products, particularly in ingestible and topical products.

Cooling compounds, that is, chemical compounds that impart a cooling sensation to the skin or the mucous membranes of the body, are well known to the art and are widely used in a variety of products such as foodstuffs, tobacco products, beverages, chewing gum, dentifrices, mouthwashes and toiletries.

A most well-known compound is 1-menthol, which is found naturally in oil of mint. Since menthol has a strong minty odor and a bitter taste, and provides a burning sensation when used in high concentrations, a variety of other menthyl ester-based and menthyl carboxamide-based cooling compounds have been developed. One that has enjoyed substantial success is N-ethyl p-menthane-carboxamide (WS-3) and is thus also often used as benchmark.

Surprisingly it has been found that certain 2-alkoxyphenols derivatives exhibit cooling intensities that are stronger than those of 1-menthol and even stronger than those of WS-3. Thus an embodiment uses at least one of the subject compounds as cooling agent, wherein the compounds are represented by formula (I)

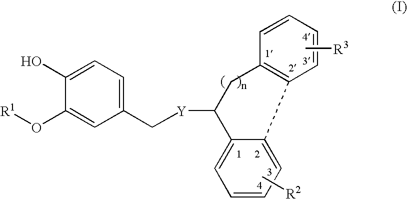

wherein

R¹ is methyl or ethyl;

Y Is, NH, O, or S;

R² and R³ are independently selected from at least one of hydrogen or C₁₋₃ alkoxy, such as methoxy, ethoxy or iso-propoxy;

n is 0 or 1; and

the dotted line between C-2 and C-2′ represents at least one of no bond, a single bond, or a —(CH₂)ₘ— group, wherein m is 1 or 2.

Non-limiting examples are compounds of formula (I) wherein R² is bonded at C-4, R³ is bonded at C-4′, or R² and R³ are bonded at C-4 and C-4′ respectively. Non-limiting examples also include compounds of formula (I) wherein R²and R³ have the same chemical formula.

In particular, embodiments are compounds of formula (I) comprising at least one of 4-((benzhydrylamino)methyl)-2-methoxyphenol, 4-((bis(4-methoxyphenyl)-methylamino)-methyl)-2-methoxyphenol, 4-((1,2-diphenylethylamino)methyl)-2-methoxyphenol, 4-((benzhydryloxy)methyl)-2-methoxyphenol, 4-((9H-fluoren-9-ylamino)methyl)-2-methoxyphenol or 4-((benzhydrylamino)methyl)-2-ethoxyphenol.

The compounds of formula (I) may be used in products that are applied to mucous membranes such as oral mucosa, or the skin, to give a codling sensation. By “applying” is meant any form of bringing into contact, for example, oral ingestion, topical application or, in the case of tobacco products, inhalation. In the case of application to the skin, it may be, for example, by including the compound in a cream or salve, or in a sprayable composition. There is therefore also provided a method of providing a cooling effect to the mucous membrane or skin by applying thereto a product comprising an effective amount of a compound as hereinabove described.

Products that are applied to the oral mucosa may include foodstuffs and beverages taken into the mouth and swallowed, and products taken for reasons other than their nutritional value, e.g. tablets, mouthwash, throat sprays, dentifrices and chewing gums. Products that are applied to the skin may be selected from perfumes, toiletries, lotions, oils and ointments, applicable to the skin of the human body, whether for medical or other reasons. Accordingly, in a further aspect there is provided a composition comprising an amount of at least one compound of formula (I) sufficient to stimulate the cold receptors in the areas of the skin or mucous membrane with which the composition comes into contact and thereby promote the desired cooling effect. A cooling effect may be achieved upon application of a product, for example, mouthwash or chewing gums, to the mucous membrane, e.g. oral mucosa, comprising less than 5000 ppm, in certain embodiments between 50 and 3000 ppm, such as about 500 ppm, of a compound of formula (I). If used for beverages the addition of about 15 ppm may be sufficient to achieve a cooling effect.

Particular examples of foodstuffs and beverages may include, but are not limited to, beverages, alcoholic or non-alcoholic, such as fruit juice beverages, fruit liquors, milk drinks, carbonated beverages, refreshing beverages, and health and nutrient drinks; frozen confectionery such as ice creams and sorbets; desserts such as jelly and pudding; confectionery such as cakes, cookies, chocolates, and chewing gum; jams; candles; breads; tea beverages such as green tee, black tea, chamomile tea, mulberry leaf tea, Roobos tea, peppermint tea; soaps; seasonings; instant beverages; snack foods and the like.

Further examples of topical products may include, but are not limited to, skin-care cosmetics, such as cleansing tissues, talcum powders, face creams, lotions, tonics and gels, hand creams, hand- and body lotions, anticellulite/slimming creams and -lotions, lotions, balms, gels, sprays and creams; sunburn cosmetics including sunscreen lotions, balms, gels, sprays and creams; after sun lotions, sprays and creams; soaps, toothpicks, lip sticks, agents for bathing, deodorants and antiperspirants, face washing creams, massage creams, and the like.

Thus there is further provided an end-product selected from at least one of products that are applied to the oral mucosa or products that are applied to the skin, such as topical products, oral care products, nasal care products, toilet articles, ingestible products and chewing gum, and the like, the end-product comprises a product base and an effective amount of at least one cooling compound of formula (I) as defined herein above.

The compounds as hereinabove described may be used alone or in combination with other cooling compounds known in the art, e.g. menthol, menthone, isopulegol, N-ethyl p-menthanecarboxamide (WS-3), N,2,3-trimethyl-2-isopropylbutanamide (WS-23), menthyl lactate, menthone glycerine acetal (Frescolat® MGA), mono-menthyl succinate (Physcool®), mono-menthyl glutarate, O-menthyl glycerine (CoolAct® 10) and 2-sec-butylcyclohexanone (Freskomenthe®), menthane, camphor, pulegol, cineol, mint oil, peppermint oil, spearmint oil, eucalyptus oil, 3-1- menthoxypropane-1,2-diol, 3-1-menthoxy-2-methylpropane-1,2-diol, p-menthane-3,8-diol, 2-1-menthoxyethane-1-ol, 3-1-menthoxypropane-1-ol, and 4-1-menthoxybutane-1-ol. Further examples of cooling compounds can be found e.g. in WO 2005/049553, which is incorporated by reference.

The cooling compounds may be employed into the products simply by directly mixing the compound with the product, or they may, in an earlier step, be entrapped with an entrapment material such as polymers, capsules, microcapsules and nanocapsules, liposomes, film formers, absorbents such as cyclic oligosaccharides, or they may be chemically bonded to a substrate, which are adapted to release the cooling compound upon application of an external stimulus such as temperature, enzyme or the like, and then mixed with the product. Or they may be added while being solubilized, dispersed, or diluted using alcohols or polyhydric alcohols, such as, glycerine, propylene glycole, triazethine and mygliol, natural gums such as gum Arabic, or surfactants, such as glycerine fatty acid esters and saccharide fatty acid esters.

The class of compounds as hereinabove described has never been described in literature and are thus novel in its own right.

Thus an embodiment further refers to a compound of formula (I)

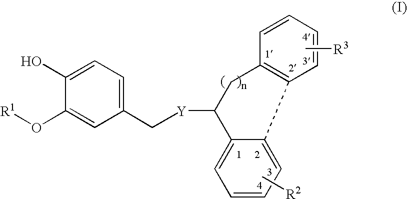

wherein

R¹ is methyl or ethyl;

Y is, NH, O, or S;

R² and R³ independently selected from at least one of hydrogen or C₁₋₃ alkoxy, such as methoxy, ethoxy or iso-propoxy;

n is 0 or 1; and

the dotted line between C-2 and C-2′ represents at least one of no bond, a single bond, or a —(CH₂)ₘ— group, wherein m is 1 or 2.

The compounds of formula (I) wherein Y is NH can be prepared by the reaction of the appropriate diphenylmethanamine with the appropriated 3-alkoxy-4-hydroxy-benzaldehyde resulting in the corresponding imine, which is then further reduced resulting in a secondary amine.

The compounds of formula (I) wherein Y is O can be prepared by reaction of the appropriated diphenylmethanol with the appropriated 4-(hydroxymethyl)-2-alkoxyphenol.

The compounds of formula (I) wherein Y is S can be prepared by alkylation of the appropriated protected 4-(mercaptomethyl)-2-methoxyphenol with the appropriated diphenylmethyl halide.

The compositions and methods are now further described with reference to the following non-limiting examples. These examples are for the purpose of illustration only and it is understood that variations and modifications can be made by one skilled in the art. It should be understood that the embodiments described are not only in the alternative, but can be combined.

### EXAMPLE 1

### 4-((benzhydrylamino)methyl)2-methoxyphenol

A) 4-((E)(benzhydrylimino)methyl)-2-methoxyphenol

In a 250 mL flask, fitted with magnetic stirrer and Dean and Stark trap (under N₂), 10.0 g of diphenylmethanamine and 8.3 g of vanillin are dissolved in 150 mL of hexane and the mixture is heated at reflux for 3 h. At the end of the reaction, about 1 mL of water was trapped by the Dean and Strak apparatus. The mixture was oiled out in the cold and the residue was recrystallized from MTBE/Hexane to yield 8.32 g of yellowish crystals. The mother liquor was further crystallized to yield a second crop of 4.9 g of white crystals for a total yield of 76%.

¹H NMR (300 MHz; CDCl₃) δ: 8.30 (s, 1H), 7.55 (d, 1H), 7.4-7.15 (multiple, 12H), 6.92 (d, 1H), 5.87 (d, 1H), 5.58 (s, 1H), 3.96 (s, 3H)

¹³C NMR (75 MHz; CDCl₃) δ: 200.6, 160.95, 144, 138.3, 127.7, 113.8. 56.0

B) 4-((benzhydrylamino)methyl)-2-methoxyphenol

In a 250 mL flask, fitted with magnetic stirrer (under N2), 8.3 g of 4-((benzhydrylimino)-methyl)-2-methoxyphenol was dissolved in 150 mL of methanol and 1.7 g of sodium borohydride were added and stirred at room temperature for overnight. The orange solution was concentrated and partitioned twice between MTBE and brine. The organic layers were washed with brine, dried over MgSO4 and concentrated. The residue was recrystallized in MTBE to give 7.80 g of white crystals having a melting point of 115-118° C. (93% yield).

¹H NMR (300 MHz; CDCl₃) δ: 7.43 (d, 4H), 7.32 (t, 4H), 7.3-7.2 (multiple, 2H), 6.85-6.75(multiple, 3H), 5.58 (br. s, 1H), 4.84 (s, 1H), 3.86 (s, 3H), 3.67 (s, 2H), 1.83 (br. s, 1H)

^{13 C NMR (}75 MHz; CDCl₃) δ: 146.5, 144.6, 144, 132.4, 128.4, 127.4, 126.9, 121, 114.2, 110.9, 66.2, 55.9, 51.7

### EXAMPLE 2

### 4-((bis(4-methoxyphenyl)methylamino)methyl)-2-methoxyphenol

The procedure outlined in Example 1 & 2 is repeated with bis(4-methoxyphenyl)-methylamine resulting in 4-((bis(4-methoxyphenyl)methylamino)methyl)-2-methoxyphenol.

¹H NMR (CDCl₃, 300 MHz) δ in ppm: 7.4 (m, 4H), 6.9-6.7 (m, 7H), 4.7 (s, 2H), 3.9 (s, 3H), 3.8 (s, 6H), 3.5 (s, 2H)

¹³C NMR (CDCl₃, 75 MHz) δ in ppm: 158.6, 146.5, 144.6, 136.5, 130.3, 128.3, 127.9120.9, 114.2, 113.8, 110.9, 64.9, 55.9, 55.2, 51.6

MS (El): 255, 243, 242, 227, 212, 198, 184, 169, 152, 135, 109, 94, 77

### EXAMPLE 3

### 4-((1,2-diphenylethylamino)methyl)-2-methoxyphenol

The procedure outlined in Example 1 is repeated with 1,2-diphenylethanamine resulting in 4-((1,2-diphenylethylamino)methyl)-2-methoxyphenol.

¹H NMR (CDCl₃, 300 MHz) δ in ppm: 7.3-7.1 (m, 10H), 6.8 (d, 1H), 6.6 (m, 2H), 3.8 (dd, 1H), 3.7 (s, 3H), 3.5 (d, 1H), 3.4 (d, 1H), 2.9 (m, 2H)

¹³C NMR (CDCl₃, 75 MHz) δ in ppm: 146.4, 144.5, 143.7, 138.9, 132.3, 129.3, 128.4, 128.3, 127.4, 127.1, 126.3, 120.8, 114.1, 110.5, 63.3, 55.8, 49.5,45.2,

MS (El): 242, 196, 181, 165, 137, 122, 106, 91, 77

### EXAMPLE 4

### 4-((benzhydryloxy)methyl)-2-methoxyphenol

In a 500 mL flask, fitted with magnetic stirrer (under N2), 9.21 g of benzhydrol, 7.70 g of vanillyl alcohol and 27.91 g of cerium ammonium nitrate are dissolved in 100 mL of acetonitrile. The mixture is stirred for 4 hours at reflux. The reaction mixture was partitioned between MTBE and water. The organic layer was washed with water and brine, dried over MgSO4, concentrated and purified by column chromatography to give 1.2 g of colorless oil (7.5% yield).

¹H NMR (CDCl₃, 300 MHz) δ in ppm: 7.4-7.2 (m, 10H), 6.9-6.8 (m, 3H), 5.6 (s, 1H), 5.4 (s, 1H), 4.5 (s, 2H), 3.9 (s, 3H)

¹³C NMR (CDCl₃, 75 MHz) δ in ppm: 146.5, 145.2, 142.2, 130.2, 128.4, 127.4, 127.1, 121.1, 114.1, 110.7, 82.1, 70.5, 55.9

MS (El): 242, 228, 213, 195, 167, 165, 138, 137

### Example 5

### 4-((9H-fluoren-9-ylamino)methyl)-2-methoxyphenol

The procedure outlined in Example 1 is repeated with 9H-fluoren-9-amine and vanillin resulting in 4-((9H-fluoren-9-ylamino)methyl)-2-methoxyphenol (mp: 148-150° C.).

¹H NMR (CDCl₃, 300 MHz) δ in ppm: 7.7 (d, 2H), 7.6 (d, 2H), 7.3 (m, 4H), 6.8 (m, 3H), 5.0 (s, 1H), 3.9 (s, 3H), 3.4 (s, 2H)

¹³C NMR (COCl₃, 75 MHz) δ in ppm: 146.4, 145.6, 144.6, 140.8, 132.6, 128.1, 127.3, 124.8, 120.9, 119.9, 114.1, 110.9, 63.2, 55.9, 48.4

MS (El): 317, 207.193, 180, 165, 152, 137, 123, 106, 94

### EXAMPLE 6

### 4-((benzhydrylamino)methyl)-2-ethoxyphenol

The procedure outlined in Example 1 is repeated with 3-ethoxy-4-hydroxybenzaldehyde resulting in 4-((benzhydrylamino)methyl)-2-ethoxyphenol (mp: 89-91° C.);

¹H NMR (CDCl₃, 300 MHz) δ in ppm: 7.4 (d, 2H), 7.3-7.1 (m, 4H), 6.9-6.7 (m, 3H), 5.6 (s, 1H), 4.8 (s, 1H), 4.1 (dd, 2H), 3.6 (s, 2H), 1.8 (s, 1H), 1.4 (t, 3H)

¹³C NMR (CDCl₃, 75 MHz) δ in ppm: 145.7, 144.7, 144.0, 132.4, 128.5, 127.4, 127.0, 120.9, 114.1, 111.8, 66.2, 64.5, 51.7. 14.9

MS (El): 331, 316, 256, 194, 182, 167, 151, 123, 106, 94, 77

### EXAMPLE 7

### Cooling Intensity

A small group of panelists was asked to taste various aqueous solutions of compounds of formula (I) and indicate which solutions had a cooling intensity similar to or slightly higher than that of a solution of menthol at 2 ppm. The results are shown in Table 1.

TABLE 1

| Chemical | Concentration | Odor |
|---|---|---|
| Comparison: | 2.0 ppm | Minty |
| I-Menthol | | |
| Comparison: | 1.5 ppm | None |
| N-ethyl p-menthanecarboxamide (WS-3) | | |
| 4-((benzhydrylamino)methyl)-2- | 0.5 ppm | None |
| methoxyphenol | | |
| 4-((benzhydryloxy)methyl)-2- | 1.0 ppm | None |
| methoxyphenol | | |

As can be seen from the results above the compounds of the present invention are at least 2 times stronger than 1-menthol and also stronger than WS-3.

### EXAMPLE 8

### Application in Toothpaste

| | | |
|---|---|---|
| Opaque toothgel | 99.50 | g |
| Compound of example 4 as a 10% solution in Ethanol | 0.10 | g |
| Peppermint oil, Terpeneless | 0.25 | g |
| Saccharin | 0.20 | g |

The chemicals are mixed in the toothgel, a piece of toothgel is put on a toothbrush and a panelist's teeth are brushed. The mouth is rinsed with water and the water is spat out. An intense cooling sensation is felt by the panelist in all areas of the mouth, with a slight bitterness. The cooling perception lasts for 45 minutes.

1. A compound of formula (I) 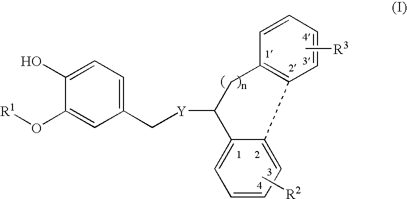
wherein
R¹ is methyl or ethyl;
Y is, NH, O, or S;
R² and R³ are independently selected from at least one of hydrogen, or C₁₋₃ alkoxy;
n is 0 or 1; and
the dotted line between C-2 and C-2′ represents at least one of no bond, a single bond, or a —(CH₂)ₘ— group, wherein m is 1 or 2.

2. The compound according to claim 1 comprising at least one of
4-((benzhydrylamino)methyl)-2-methoxyphenol,
4-((bis(4-methoxyphenyl)-methylamino)-methyl)-2-methoxyphenol,
4-((1,2-diphenylethylamino)methyl)-2-methoxyphenol,
4-((benzhydryloxy)methyl)-2-methoxyphenol,
4-((9H-fluoren-9-ylamino)methyl)-2-methoxyphenol or
4-((benzhydrylamino)methyl)-2-ethoxyphenol.

3. (canceled)

4. A method of providing a cooling effect to the mouth or skin by applying thereto a product comprising a compound of formula (I) as defined in claim 1.

5. A product selected from at least one of products that are applied to the oral mucosa or products that are applied to the skin, the product comprising a product base and an effective amount of a cooling compound of formula (I) as defined in claim 1.

6. The compound of claim 1, wherein R² is bonded at C-4 or R³ is bonded at C-4′, or R² and R³ are bonded at C-4 and C-4′, respectively.

7. The compound of claim 1, wherein R² and R³ have the same chemical formula.

8. The method of claim 4, wherein the compound comprises R² bonded at C-4 or R³ bonded at C-4′, or R² and R³ bonded at C-4 and C-4′, respectively.

9. The method of claim 4, wherein R² and R³ of the compound have the same chemical formula.

10. The method of claim 4, wherein the compound comprises at least one of
4-((benzhydrylamino)methyl)-2-methoxyphenol,
4-((bis(4-methoxyphenyl)-methylamino)-methyl)-2-methoxyphenol,
4-((1,2-diphenylethylamino)methyl)-2-methoxyphenol,
4-((benzhydryloxy)methyl)-2-methoxyphenol,
4-((9H-fluoren-9-ylamino)methyl)-2-methoxyphenol or
4-((benzhydrylamino)methyl)-2-ethoxyphenol.

11. The method of claim 4, wherein the compound is used in combination with one or more additional cooling compounds.

12. The method of claim 11, wherein the one or more additional cooling compounds is at least one of of menthol, menthone, isopulegol, N-ethyl p-menthanecarboxamide, N,2,3-trimethyl-2-isopropylbulanamide, menthyl lactate, menthone glycerine acetal, mono-menthyl succinate, mono-menthyl glutarate, O-menthyl glycerine, 2-sec-butylcyclohexanone, menthane, camphor, pulegol, cineol. mint oil, peppermint oil, spearmint oil, eucalyptus oil, 3-1-menthoxypropane-1,2-diol, 3-1-menthoxy-2-methylpropane-1,2-diol, p-menthane-3,8-diol, 2-1-menthoxyethane-1-ol, 3-1-menthoxypropane-1-ol, or 4-1-menthoxybutane-1-ol.

13. The product of claim 5, wherein the compound comprises R² bonded at C-4 or R³ bonded at C-4′, or R² and R³ bonded at C-4 and C-4′, respectively.

14. The product of claim 5, wherein R² and R³ of the compound have the same chemical formula.

15. The product of claim 5, wherein the compound comprises at least one of
4-((benzhydrylamino)methyl)-2-methoxyphenol,
4-((bis(4-methoxyphenyl)-methylamino)-methyl)-2-methoxyphenol,
4-((1,2-diphenylethylamino)methyl)-2-methoxyphenol.
4-((benzhydryloxy)methyl)-2-methoxyphenol,
4-((9H-fluoren-9-ylamino)methyl)-2-methoxyphenol or
4-((benzhydrylamino)methyl)-2-ethoxyphenol.

16. The product of claim 5, wherein the product comprises less than 5,000 ppm of the compound.

17. The product of claim 5, wherein the product comprises an amount of from 50 to 3,000 ppm of the compound.

18. The product of claim 5, wherein the product comprises a beverage comprising about 15 ppm of the compound.

19. The product of claim 5, wherein the product comprises a foodstuff beverage, chewing gum, or tobacco product.

20. The product of claim 5, wherein the product comprises a topical product, an oral care product, a nasal care product, or a toilet article.

21. The product of claim 5, wherein the cooling compound is incorporated into the product base by direct mixing with the product base; by entrapment with a polymer, capsule, microcapsule or nanocapsule, liposome, film former, or absorbent prior to mixing with the product base; by chemical bonding to a substrate adapted to release the cooling compound upon application of an external stimulus, prior to mixing with the product base; or by adding the cooling compound while solubilized, dispersed, or diluted using alcohols, polyhydric alcohols, or surfactants.
